(19) United States
(12) Patent Application Publication
(10) Pub. No.: US 2003/0045922 A1
(43) Pub. Date: Mar. 6, 2003

(54) Skin treatment method and apparatus

(76) Inventor: Nancy Northrop, Kennett Square, PA (US)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 09/942,324

(22) Filed: Aug. 29, 2001

(51) Int. Cl. A61N 1/04

(52) U.S. Cl. 607/139

(57) ABSTRACT

A method of treating the face includes positioning electrode pairs so as to stimulate predetermined nerves on a first side and a second side of the face and alternately stimulating a first pair and a second pair of the electrode pairs. The first pair is located on the first side of the face and the second pair comprises a reciprocal electrode pair of the first pair and is located on the second side of the face. The first pair and the second pair are stimulated alternately for a first predetermined period of time.

### FIELD OF THE INVENTION

The present invention relates to a method and apparatus for improving facial contour and diminishing fine lines and wrinkles by electrical stimulation of the nerves, muscles and/or skin of the face.

### BACKGROUND OF THE INVENTION

The use of neuromuscular electrical stimulation (“NMES”) is known for preventing atrophy of muscles. These devices employ electrodes positioned on a user's body for delivering an electrical current through the skin to stimulate muscles.

NMES is also known for stimulating the muscles of the face. Many such devices comprise hand-held devices for electrically stimulating the facial muscles. The hand-held devices typically have an electrode pair that is held against the skin of the face in order to deliver an electrical impulse through the skin. However, such devices suffer from the drawback that the user of the device must determine the placement of the electrodes on the skin. Thus, without significant training, an individual will typically have difficulty in properly placing the electrode pair on the skin to stimulate particular facial muscles or nerves in accordance with a treatment regimen. Placement of the electrodes greatly affects the efficacy of the treatment.

Masks carrying electrodes for stimulating the muscles of the face are also known. Such masks typically include single electrodes distributed across the entire face. These devices provide little adjustability to the contour of different user's faces. Those devices that do provide some adjustability require a significant amount of judgment to be exercised in placing the electrodes with respect to the nerves of the particular user's face. In addition, these masks do not employ pairs of electrodes for stimulating the facial nerves in a regimen for treating the skin of the entire face to improve facial contour, health and vitality and to diminish fine lines and wrinkles.

Thus, despite some advances in treating the face to improve facial contour and diminish fine lines and wrinkles, there is a need for a method and apparatus for treating the nerves, muscles, and skin of the face in a reproducible, efficient and effective regimen.

### SUMMARY OF THE INVENTION

In one aspect of the present invention, a method of treating the nerves, muscles, and/or skin of the face comprises positioning electrode pairs so as to stimulate predetermined nerves on a first side and a second side of the face and alternately stimulating a first pair and a second pair of the electrode pairs. The first pair is located on the first side of the face and the second pair comprises a reciprocal electrode pair of the first pair and is located on the second side of the face. The first pair and the second pair are stimulated alternately for a first predetermined period of time.

The first pair is desirably stimulated a second predetermined period of time and, after the first pair is stimulated, the second pair is stimulated for a third predetermined period of time. The second period of time and the third period of time, preferably, are the same.

After the first predetermined period of time has elapsed, a third pair and a fourth pair are preferably alternately stimulated for a fourth predetermined period of time. The third pair comprises an electrode pair on the first side of the face and the fourth pair comprises a reciprocal electrode pair of the third pair, located on the second side of the face.

After the fourth period of time has elapsed, a fifth pair and a sixth pair are preferably alternately stimulated for a fifth predetermined period of time. The fifth pair comprises an electrode pair on the first side of the face and the sixth pair comprises a reciprocal electrode pair of the fifth pair, located on the second side of the face.

The first predetermined period of time and the second predetermined period of time may, for example, each comprise one second. The third predetermined period of time may, for example, comprise three minutes.

The pattern for activating the electrodes reciprocally stimulates the facial muscles on either side of the face to cut the total treatment time by 50%. For example, a regimen of reciprocating, bilateral facial stimulation with a 1:1 “duty cycle” (1 second of stimulation followed by 1 second of rest) completes a protocol of 90 repetitions of each of 12 facial motor points in only 18 minutes. Without this pattern for activating the electrodes, the total treatment time required to elicit 90 repetitions of 12 motor points in a 1:1 duty cycle would be 36 minutes.

Preferably, the electrode pairs of at least two of the electrode pairs share a common electrode.

The electrode pairs desirably comprise six electrode pairs on the first side of the face, with nine electrodes being used for the six electrode pairs. At least two pairs of the electrodes pairs may be used to stimulate the same nerves. In addition, at least one pair of the electrode pairs may be used to stimulate more than one nerve.

The electrode pairs are preferably used to stimulate the Zygomatic, Temporal, Buccal, Mandibular, and Cervical nerve branches. One of the pairs preferably stimulates the Zygomatic and Buccal nerve branches, whereas other of the pairs stimulate the Mandibular and Cervical nerve branches.

In another aspect of the present invention, an apparatus for treating the nerves, muscles, and/or skin of the face comprises a plurality of members shaped generally to the contour of the face and a plurality of electrode pairs. At least one of the electrode pairs is mounted on each of the members. The members are connected to one another and adjustable with respect to one another so as to conform to the facial contour of different users. At least one of the electrodes can be mounted at a plurality of positions on the members and locked in one of the positions.

Variably positionable electrodes enable apparatuses in accordance with embodiments of the invention to adjust to the faces of different users. There are variations in facial neuro-anatomy from person to person. Therefore, optimal electrode placement is necessary in order to elicit the desired neuromuscular response in each individual user of the device. This design feature allows the device to be custom fit to each face, thereby facilitating precise electrode placement and neuromuscular response.

The members preferably comprise first members for a first side of the face and second members for a second side of the face.

Two of the electrode pairs may be comprised of three electrodes. One of the three electrodes may be a member of two electrode pairs.

Three of the electrode pairs may be comprised of four electrodes. One of the four electrodes may be a member of three electrode pairs.

The first members, in certain embodiments, carry nine electrodes.

At least two of the electrode pairs may be used to stimulate the same nerves. The electrode pairs of the first members preferably stimulate the Zygomatic and Buccal nerve branches.

The electrode pairs of the first members preferably stimulate the Mandibular and Cervical nerve branches. The electrode pairs of the first members may comprise six electrode pairs for a first side of the face and the electrode pairs preferably stimulate the Zygomatic, Temporal, Buccal, Mandibular, and Cervical nerve branches.

In a further aspect of the present invention, an apparatus for treating the nerves, muscles, and/or skin of the face comprises a structure having at least one member attachable to the face and a plurality of electrodes. Pairs of the electrodes are arranged with respect to predetermined facial points for stimulating the nerves of the face. The electrodes include a shared electrode being a member of more than one of the pairs. The shared electrode stimulates more than one of the facial points.

This feature enables the apparatus to multiplex the electrical signal with fewer electrode pairs than would be necessary to cover 12 facial motor points if one positive and one negative electrode were used for each pair. A common electrode with specific polarity completes a pair for more than one electrode pair. A common electrode with specific polarity enables the treatment of multiple facial motor points to occur in less time than if one positive and one negative electrode were used for each pair.

### BRIEF DESCRIPTION OF THE DRAWINGS

These and other features, aspects and advantages of the present invention will become better understood with regard to the following description, appended claims, and accompanying drawings where:

FIG. 1 is a schematic, side elevation view of an apparatus for treating the skin of the face in accordance with an embodiment of the invention;

FIG. 2 is a front elevation view of the apparatus in accordance with the embodiment of FIG. 1;

FIG. 3 is a side elevation view of the apparatus in accordance with the embodiment of FIGS. 1 and 2;

FIG. 4 is a partial side elevation view of the apparatus in accordance with the embodiment of FIGS. 1-3;

FIG. 5 is a schematic, front-left perspective view showing the top side of a control unit for the apparatus in accordance with the embodiment of FIGS. 1-4;

FIG. 6 is a schematic diagram of the control unit connected to the electrode structure in an apparatus in accordance with the embodiment of FIGS. 1-5;

FIG. 7 is a schematic diagram of user controls for the apparatus in accordance with the embodiment of FIGS. 1-6;

FIG. 8 is a graph illustrating an output waveform for the control unit of the apparatus in accordance with the embodiment of FIGS. 1-7; and

FIGS. 9a and 9b are front and cross-sectional views of the electrode structure in accordance with the embodiment of FIGS. 1-7.

### DETAILED DESCRIPTION

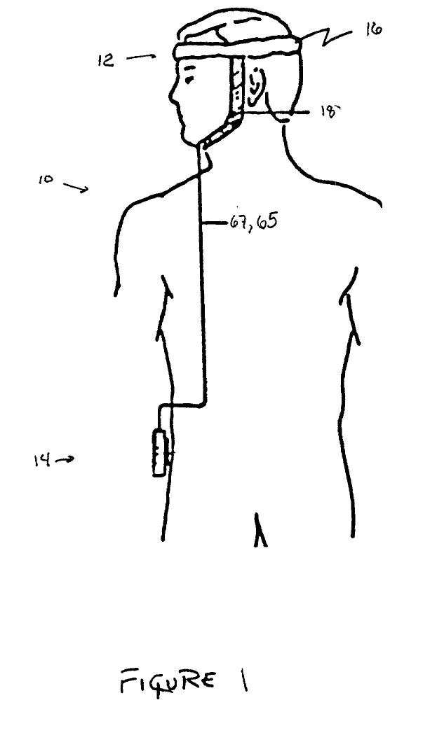

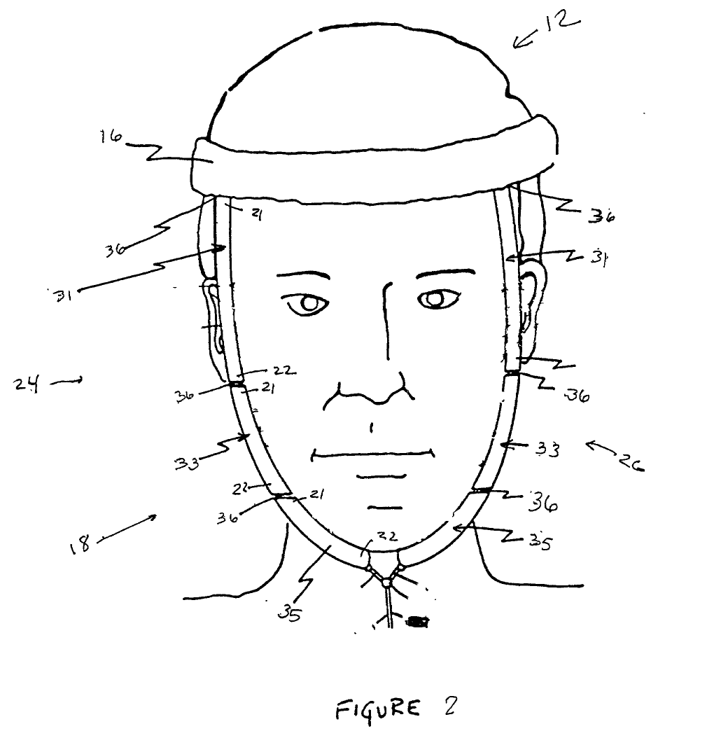

An apparatus for treating the nerves, muscles and/or skin of the face in accordance with an embodiment of the present invention is shown in FIGS. 1-9. The apparatus 10 includes a headgear 12 to be worn on a user's head and a control unit 14 for controlling the treatment applied to the user's face via the headgear 12, as shown in FIG. 1. The headgear 12, as shown in FIG. 2, comprises a structure that is supported by the user's head and face. In certain preferred embodiments, the headgear 12 includes a support 16 worn by the user on the user's head. The headgear 12 also includes an electrode structure 18 that is conformable to the contour of the outline of a user's face. The electrode structure 18 preferably comprises a plurality of arcuate members, each having a first end 21 and a second end 22 that are joinable to ends of the other arcuate members and/or the support 16 so as to lay upon and encircle the user's face. As shown in FIG. 2, the electrode structure 18 comprises a left side structure 24 and a right side structure 26. Preferably, the left side structure 24 and the right side structure 26 each comprise a plurality of arcuate members. For example, in the embodiment shown in FIG. 2, the left side structure 24 and the right side structure 26 each comprise three arcuate members including a first arcuate member 31, a second arcuate member 30 and a third arcuate member 35. A first end 21 of the first arcuate member 31 is attached to the head support 16. The first arcuate member 31 extends on the user's face, overlying the temple of the user. The first arcuate member 31 terminates in a second end 22 that is attached to a first end 21 of second arcuate member 33. The second arcuate member 33 extends on the user's face, generally overlying the cheek and beside the mouth of the user, terminating in a second end 22. The second end 22 of the second arcuate member 33 is attached to a first end 21 of the third arcuate member 35. The third arcuate member 35 extends on the user's face, overlying one side of the chin, terminating in a second end 22. The second end 22 of the third arcuate member 35 is connected to the third arcuate member on the right side structure 26. The right side structure 26 is preferably similarly constructed, including a first, second and third arcuate member having ends attached to one another with an end of one of the arcuate members being attached to the head support 16. Although three arcuate members are shown, the number of arcuate members is not critical to the invention.

Preferably, each end of each arcuate member is attached to another end of an arcuate member, or to the head support, by an adjustable attachment structure 36, which allows the headgear 12 to conform to the contour of the faces of different users. In preferred embodiments, the adjustable attachment structures 36 are used to generally shorten or generally lengthen the left side structure 24 and right side structure 26, to conform to the particular user's facial contour. In the illustrated embodiment, each of the adjustable attachment structures 36 comprise a telescoping mechanism that can be used to bring its associated arcuate arms closer and farther away from each other, as desired, to cause the headgear 12 to conform to the user's face.

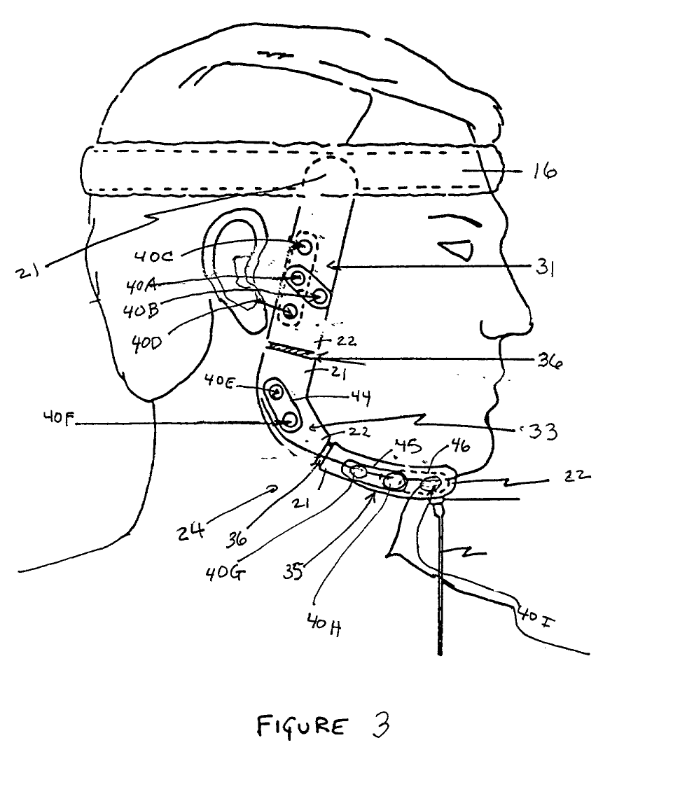

FIG. 3 illustrates the electrodes carried by each of the arcuate members of the electrode structure 18. FIG. 3 shows the left side structure 24 and the electrodes carried thereon. The right side structure 26 is preferably similarly constructed with corresponding electrodes arranged on arcuate members corresponding to the arcuate members of the left side structure 24.

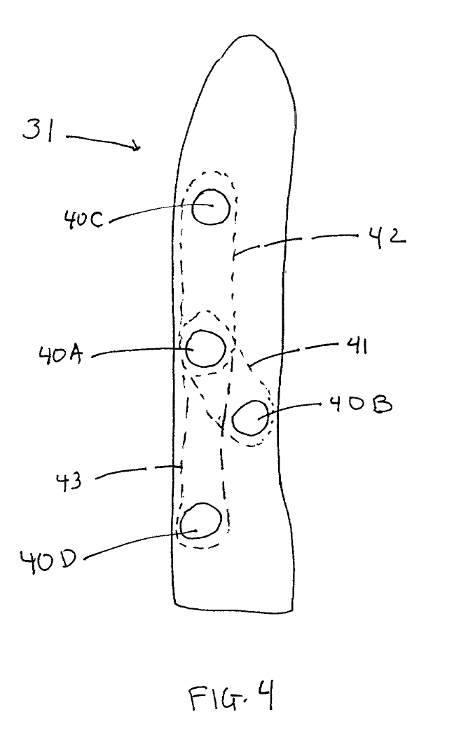

The left side structure 24 carries electrodes as shown in FIG. 3. The electrodes are arranged in electrode pairs and are preferably arranged so that at least one electrode (referred to herein as a “common electrode”) is a member of more than one electrode pair. For example, the first arcuate member 31 carries four electrode that establish three electrode pairs. The first arcuate member 31 carries electrodes 40A, 40B, 40C and 40D. These electrodes are arranged so as to comprise three electrode pairs. As shown in FIG. 4, the first arcuate member 31 has a first electrode pair 41, comprising electrode 40A and electrode 40B. The second electrode pair 42 comprises electrode 40A and electrode 40C. The third electrode pair 43 comprises electrode 40A and electrode 40D. Electrode 40A is a common electrode in that it is shared by the three electrode pairs 41, 42, 43 on the first arcuate member 31. Thus, the electrodes on the first arcuate member 31 may be activated in three separate pairs while requiring only four electrodes on the first arcuate member 31.

Referring back to FIG. 3, the second arcuate member 33 carries electrode 40E and electrode 40F, comprising a fourth electrode pair 44. The third arcuate member 35 carries electrode 40G, electrode 40H and electrode 40I. These three electrodes comprise two electrode pairs so that electrode 40H is shared between two electrode pairs. The fifth electrode pair 45 comprises electrode 40G and electrode 40H. The sixth electrode pair 46 comprises electrode 40H and electrode 40I. Thus, the third arcuate member 35 carries only three electrodes for establishing two electrode pairs 45, 46.

In the embodiment shown in FIG. 3, nine electrodes are used. However, the number of electrodes is not critical to the invention. Further, the electrodes may be arranged in any number of pairs that share one or more electrodes so as to efficiently utilize a minimum number of electrodes to stimulate the desired facial points. However, the feature of a common electrode is preferred in that it allows the apparatus 10 to multiplex the electrical signal with fewer electrode pairs than would be necessary to cover 12 facial motor points if a unique positive electrode and a unique negative electrode were used in each pair to complete a given circuit. Further, as will be described in greater detail below, the feature of a common electrode enables the treatment of multiple facial points to occur in less time than if a unique positive electrode and a unique negative electrode were used in each pair to complete a given circuit. In the illustrated embodiment, common electrodes are provided at particular locations to achieve advantages in addition to those described above. For example, the common electrode 40A is placed over the insertion of the facial nerve, which innervates the Zygomatic, Temporal, Buccal, Mandibular, and Cervical nerve branches. Placing common electrode 40A at the insertion of the facial nerve and completing its circuit with electrodes placed at distal anatomical placements along its specific branches facilitates more efficient and complete recruitment of the specific nerves and muscles of the face.

The electrodes preferably have a surface that is exposed on the arcuate members so as to contact the skin of the user. Desirably, a conductive medium is applied to the exposed surfaces of the electrodes.

Preferably, the electrodes are positioned on the arcuate members so that, once the adjustable attachment structures 36 are used to conform the left side structure 24 and right side structure 26 to the user's face, the electrode pairs carried by the arcuate members correspond to the facial nerve branches discussed above. Thus, the headgear 12 may be structured to require little adjustment of the electrodes on the arcuate members, once the size of the headgear 12 is adjusted to the user's face. In these embodiments, the headgear 12 provides a reproducible and reliable means for treating a particular user's face.

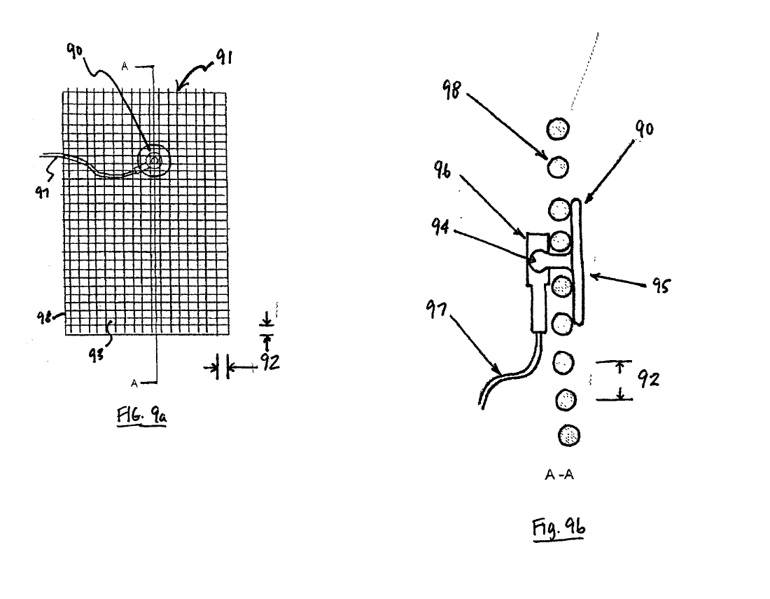

In other preferred embodiments, the electrodes can be mounted on the arcuate members at a plurality of positions and locked in one of the positions to provide reproducible and reliable treatment. The variably positionable electrodes may be adjusted by the user but preferably by a trained professional to ensure accurate stimulation of the nerve branches discussed above. Therefore, little judgment is required by the particular user in reproducibly performing the treatment regimen. FIGS. 9a and 9b show an apparatus for mounting the electrodes in the illustrated embodiment. Each electrode 90 is interfaced with the arcuate members of the headgear with a net-like structure 91, preferably comprised of threads 98 of synthetic material, having a pitch 92 sufficient to create openings 93 large enough through which to push the post 94 of a snap electrode 90. In this way, each electrode 90 can be positioned with its face side 95 anywhere on the user's face where there is an arcuate member, and the corresponding connector snap 96 can be snapped onto the post 94 of the electrode 90 to secure the electrical connection between the lead wire 97 and the electrode 90. It should be understood that the structure permits easy relocation of the electrodes 90 by unsnapping the connector snap 96 from the post 94, moving the post 94 to a different opening 93 on the net 91, and re-snapping the connector snap 96 onto the post 94. Of course, the connector snap 96 can alternatively be moved to another electrode that is already in place, if that is desirable to effect treatment. In preferred embodiments, lead wires are attached along the edge of each arcuate member, and the lead wires have free hanging ends with snaps on them, so that the lead wires are available for attachment to electrodes as needed. Also preferably, dummy snap posts can be provided to attach unused lead wires if necessary. Also preferably, the face side 95 of each electrodes 90 is comprised of stainless steel.

Each electrode pair establishes a stimulation point for stimulating the facial nerves of the user. Preferably, each electrode pair is positioned over the myoneural end plates of facial muscles, which are sites of connection between facial nerves and muscles. Preferably, passive stimulation of the muscles of facial expression is accomplished, as opposed to gross motor response of the facial muscles. Most preferably, the Temporal, Zygomatic, Buccal, Mandibular, and Cervical nerve branches are stimulated at the myoneural end plates to achieve treatment of the entire face, improving facial contour, diminishing fine lines and wrinkles, and improving the appearance of the skin of the face, overall. The inventor has found the above-discussed structure and arrangement of electrodes achieves these objectives. Accordingly, the electrode pairs of headgear 12 are preferably arranged so as to stimulate the Temporal, Zygomatic, Buccal, Mandibular, and Cervical nerve branches.

For example, the electrode pairs may be arranged as shown in FIGS. 1-4. The first electrode pair 41 is arranged to stimulate the Zygomatic nerve branch, the second electrode pair 42 is arranged to stimulate the Temporal nerve branch, and a third electrode pair 43 is arranged to stimulate the Zygomatic and preferably nerve branches. The fourth electrode pair 44 is arrange to stimulate the Mandibular and Cervical nerve branches. The fifth electrode pair 45 and sixth electrode pair 46 are arranged to stimulate the Mandibular nerve branch. Preferably, the right side structure 26 has a reciprocal arrangement, establishing six electrode pairs corresponding to those discussed above for the left side structure 24. Thus, the right side structure 26 also has six electrode pairs established by nine electrodes carried on the arcuate members.

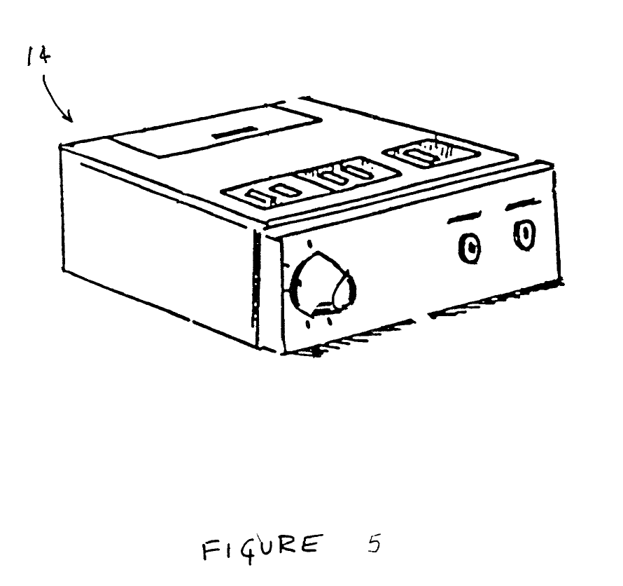

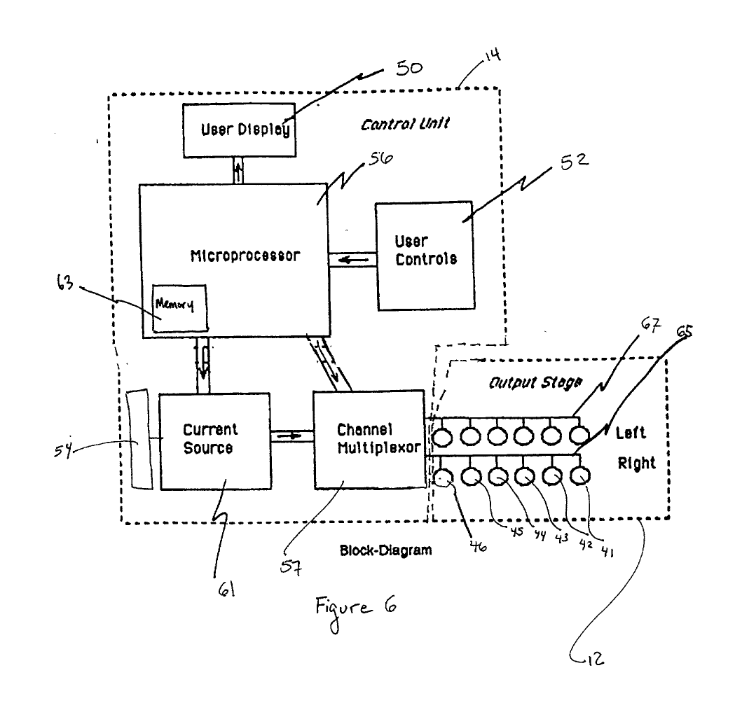

The apparatus 10 includes a control unit 14, as shown in FIGS. 5, 6, 7 and 8. A schematic of a suitable control unit 14 and its components are shown in FIG. 6. The control unit 14 preferably includes a display 50, controls 52, a power source 54, a current source 61, a microprocessor 56, and a multiplexer 57. The control unit 14 controls the operation of the electrodes arranged on the electrode structure 18. The power source 54 preferably comprises an internal power source, such as a nine volt DC battery. The power source may also comprise a rechargeable battery and a socket for connection to a conventional domestic AC wall outlet. In other alternatives, the power source 54 comprises a socket for connection to an AC wall outlet directly. The power source 54 includes a current source 61 for generating the current in a desired waveform, which will be discussed further detail below. The control unit 14 also includes a microprocessor 56 for controlling the output of the current source 61 according to the desired treatment regimen. The microprocessor 56 includes a memory, preferably comprising a rewritable, programmable read-only memory 63 for storing certain criteria for executing the treatment regimen. The control unit also includes a multiplexer 57 for switching between activation of various electrode pairs on the electrode structure 18.

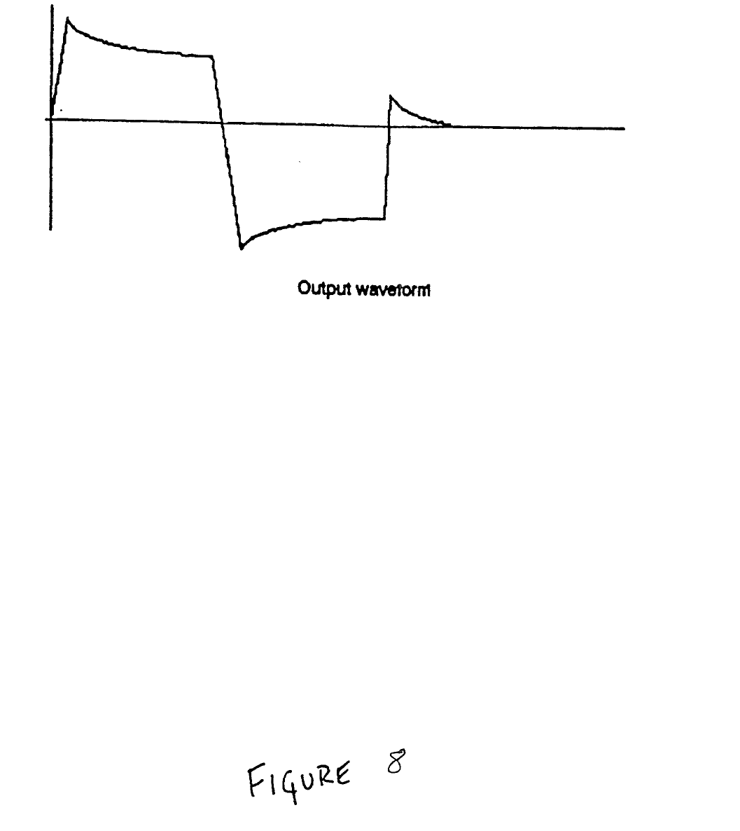

The current source 61 generates the current in the waveform shown in FIG. 8. The current increases for 0.5 seconds and then decreases for 0.5 seconds. The waveform is biphasic, having a phased duration of 400 microseconds and a repetition rate of 500 pulses per second. Referring again to FIGS. 2-6, the multiplexer 57 has six channels for the electrodes on the right side structure 26 and six channels for the electrodes on the left side structure 24. The multiplexer 57 is connected to the channels for the right side structure 26 through wire 65 and connected to the channels for the left side structure 24 through wire 67. Each channel corresponds to an electrode pair on the right side structure 26 or left side structure 24. The wire 67 for the left side structure 24 has channels connected to the electrode pairs 41, 42, 43, 44, 45 and 46 and the wire 65 for the right side structure 26 has channels for six electrode pairs, which preferably correspond to the electrode pairs for the left side structure 24.

The multiplexer 57 connects the current source 61 to one of the channels according to the desired treatment regimen. The microprocessor 56 utilizes information stored in the memory 63 to control the current source 61 and multiplexer 57 so that the electrodes of the electrode pairs on the left side structure 24 and right side structure 26 are activated in the desired treatment regimen.

The microprocessor 56 controls activation of the electrode pairs, preferably in the following regimen. A first electrode pair is coupled to the waveform generated by the current source 61 described above for a first side of the face. The first electrode pair is activated for a predetermined period of time. For example, the first electrode pair 41 on the left side structure 24 may be activated for one second. After the first electrode pair 41 on the left side structure 24 is activated for one second, the microprocessor 56 controls the apparatus so that the first electrode pair 41 is no longer activated, while a first electrode pair of the right side structure 26 is activated for one second. Preferably, the first electrode pair 41 on the left side structure 56 corresponds to the first electrode pair on the right side structure 26 so that the Zygomatic nerve branch on either side of the face is stimulated. After activating the first electrode pair on the right side structure 26 for one second, the first electrode pair 41 on the left side structure 24 is again activated while the first electrode pair on the right side structure 26 is no longer activated. Electrode pairs on each side of the face are stimulated in a reciprocating fashion, each side being stimulated for a predetermined period of time while the other side rests. Thus, the Zygomatic nerve branch on each side of the face is stimulated reciprocally, alternating between each side of the face.

The first electrode pair on each side of the face is stimulated alternately for a predetermined period of time, for example, three minutes. The microprocessor 56 then controls the apparatus so that a second electrode pair is stimulated for a predetermined period of time. For example, second electrode pair 42 of the left side structure 24 may be stimulated for one second. After such stimulation, the second electrode pair on the right side structure 26 is stimulated for a predetermined period of time while the electrode pair 42 on the left side structure 24 is no longer activated. For example, the second electrode pair on the right side structure 26 is stimulated for one second. The second electrode pairs on either side of the face are reciprocally stimulated, alternating between each side of the face for a predetermined period of time, for example, three minutes. Preferably, the second electrode pair on each side of the face stimulates the Temporal nerve branch on each side of the face. This pattern is repeated until each of the six electrode pairs on each side of the face have been stimulated. Thus, in a regimen reciprocally stimulating electrode pairs on each side of the face, activating six electrode pairs on each side of the face, for a total of twelve electrode pairs, the treatment regimen discussed above is carried out in 18 minutes.

As described above, the feature of a common electrode enables the treatment of multiple facial points to occur in less time than if a unique positive electrode and a unique negative electrode were used in each pair to complete a given circuit. In this embodiment, the pattern of electrodes facilitating bilateral facial stimulation cuts the total treatment time by 50%, from 36 minutes to 18 minutes. The pattern of electrodes built into the facial apparatus allows implementation of the described program of reciprocal bilateral facial stimulation with a 1:1 duty cycle (1 second of stimulation followed by 1 second of rest) to complete a protocol of 90 repetitions of each of 12 facial motor points in only 18 minutes. Without the described pattern of electrodes to facilitate the described program, the total treatment time required to elicit 90 repetitions of 12 motor points in a 1:1 duty cycle would be 36 minutes. It should be understood that the describe pattern is merely one embodiment, and a variety of other patterns using one or more common electrodes can be utilized to achieve a variety of time savings.

The reciprocal stimulation of the electrode pairs on each side of the face reduces the treatment time for treating the entire face. The treatment regimen incorporates a rest period for electrode pairs on each side of the face, during which a corresponding electrode pair on the opposite side of the face is stimulated. The inventor has found that reciprocally stimulating electrode pairs on either side of the face in this manner for a predetermined period of time achieves very efficient and effective treatment of the entire face for improvement of contour and diminishing fine lines and wrinkles.

Referring again to FIG. 6, the control unit 14 includes a display 50 and controls 52. In certain preferred embodiments, the display comprises an LCD showing the amperage of the current coupled to each electrode pair during activation of the electrode pairs in the treatment regimen. The display may also include a low battery indicator, an indication of the particular electrode pair being stimulated, and an indication of which side of the face is stimulated as the treatment regimen progresses. The display may also show the elapsed time of the treatment regimen.

The display may also include a compliance meter showing the user's use of the apparatus 10. For example, use of the treatment regimen described above may be recommended daily for a period of 30 days. The compliance meter would show how often the treatment regimen was executed. In embodiments having an interior power source, the display preferably includes a power indicator, which indicates when the battery must be replaced or recharged.

Preferably, the control unit 14 includes a clip or other device for attaching the control unit 14 to the user's clothing. Thus, the user may wear the apparatus 10 while moving about freely.

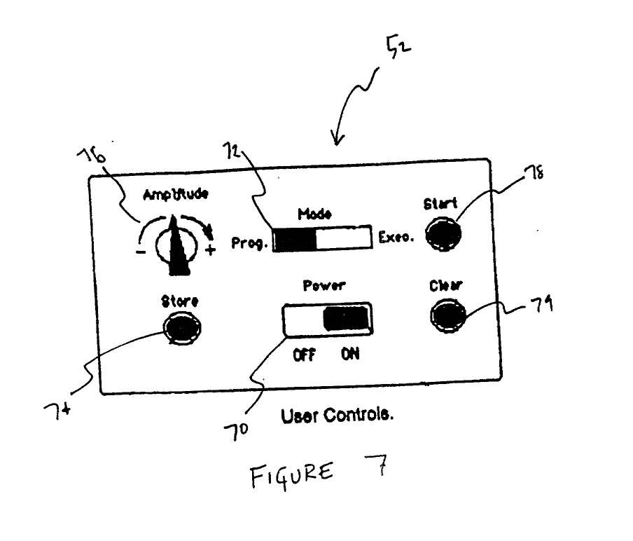

Referring to FIG. 7, the controls 52 preferably include a power switch 70 and a switch 72 for switching between the treatment mode and the programming mode. In the treatment mode, the treatment regimen described above is executed. In the programming mode, a dermatologist, technician or other professional can program the amplitude of the current utilized for each of the electrode pairs, as will be described in further detail below. The controls 52 preferably include a store button 74 for storing the amplitude values for each of the electrode pairs. The controls also include an amplitude adjustment dial 76, or keypad, for entering the amplitude values to be stored. The controls preferably include a start button 78 for starting the treatment regimen and a clear button 79 for clearing stored amplitude values.

The amplitudes for the treatment regimen are programmed into the apparatus 10, preferably by a trained practitioner. The trained practitioner fits the headgear 12 to the user's head and face so that the electrode structure 18 generally conforms to the contour of the user's face. The practitioner switches the power on and utilizes the mode button to put the apparatus 10 in the programming mode. The microprocessor 56 controls operation of the apparatus so that a first electrode pair on one side of the face is coupled to the current source 61 via the multiplexer 57. The practitioner adjusts the amplitude, using the amplitude dial or keypad, until visible muscle fasciculation of the facial muscle stimulated by that electrode pair occurs. The practitioner determines when the appropriate amplitude has been reached and presses the store button to store the amplitude value in the memory 63 of the microprocessor 56. The microprocessor then controls the apparatus to activate the corresponding electrode pair on the other side of the user's face. The practitioner selects an appropriate amplitude and stores the same in the memory 63. Thus, the memory 63 stores amplitude values for each of the electrode pairs on each of the left and right sides of the user's face. The microprocessor 56 preferably automatically carries out similar steps for each of the electrode pairs on each side of the user's face so that the practitioner will store amplitude valued for each. After all the amplitude values are stored the apparatus 10 may be switched to the treatment mode.

It should be understood that, although the invention has been described for use with a treatment of the face and the stimulation of facial nerves, the invention can be used to treat other parts of the body and to stimulate the muscles, skin, and/or nerves of other parts of the body, such as, for example, arm, leg, neck, back, stomach, chest, etc. For example, the electrodes described herein can be mounted on other body surfaces using the described or other mounting apparatuses, and stimulation according to the methods described herein can be applied. The same benefits described herein for treatment of the face can be achieved for other parts of the body by using the invention in such a manner, including but not limited to time-saving benefits and placement of common electrode benefits (e.g., placement of a common electrode on an insertion of a nerve leading to a plurality of nerve branches).

Further, it should be understood that although the invention has been described for use in improving facial contour and diminishing fine lines and wrinkles by electrical stimulation of the nerves, muscles and/or skin of the face, the invention is not limited to such purposes or uses. For example, stimulation of other body nerves can achieve these and other results for other parts of the body. Such achievable results include muscle and skin toning and nerve stimulation. It should be understood that the predetermined times discussed herein can be tailored for each desired application to achieve the most optimum results.

Further, it should be understood that the invention is not limited for use with human bodies, but the devices and methods described herein can be adapted for use with animals to, for example, enhance or supplement veterinary treatments. [0061] Finally, although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A method of treating a face, comprising:
positioning electrode pairs so as to stimulate predetermined nerves on a first side and a second side of the face; and
alternately stimulating a first pair and a second pair of said electrode pairs, said first pair being located on the first side of the face and said second pair comprising a reciprocal electrode pair of said first pair and being located on the second side of the face, said first pair and said second pair being stimulated alternately for a first predetermined period of time.

2. The method of claim 1, wherein said first pair is stimulated a second predetermined period of time and, after said first pair is stimulated, said second pair is stimulated for a third predetermined period of time.

3. The method of claim 1, wherein after said first predetermined period of time has elapsed, a third pair and a fourth pair are alternately stimulated for a fourth predetermined period of time, said third pair comprising an electrode pair on the first side of the face and said fourth pair comprising a reciprocal electrode pair of said third pair and being located on the second side of the face.

4. The method of claim 3, wherein after said fourth period of time has elapsed, a fifth pair and a sixth pair are alternately stimulated for a fifth predetermined period of time, said fifth pair comprising an electrode pair on the first side of the face and said sixth pair comprising a reciprocal electrode pair of said fifth pair and being located on the second side of the face.

5. The method of claim 1, wherein said first predetermined period of time and said second predetermined period of time each comprise one second.

6. The method of claim 5, wherein said third predetermined period of time comprises three minutes.

7. The method of claim 1, wherein said electrode pairs of at least two of said electrode pairs share a common electrode.

8. The method of claim 7, wherein said electrode pairs comprise six electrode pairs on the first side of the face, nine electrodes being used for said six electrode pairs.

9. The method of claim 1, wherein at least two pairs of said electrode pairs stimulate the same nerves.

10. The method of claim 9, wherein at least one pair of said electrode pairs stimulates more than one nerve.

11. The method of claim 1, wherein said electrode pairs stimulate the Zygomatic, Temporal, Buccal, Mandibular, and Cervical nerve branches.

12. The method of claim 11, wherein one of said pairs stimulates the Zygomatic and Buccal nerve branches.

13. The method of claim 12, wherein another of said pairs stimulates the Mandibular and Cervical nerve branches.

14. An apparatus for treating a face, comprising:
a plurality of members shaped generally to the contour of the face; and
a plurality of electrode pairs, at least one of said electrode pairs being mounted on each of said members;
said members being connected to one another and adjustable with respect to one another so as to be conformable to the facial contours of different users, at least one of said electrodes being mountable at each of a plurality of positions on said members.

15. The apparatus of claim 14, wherein said members comprise first members for a first side of the face and second members for a second side of the face.

16. The apparatus of claim 14, wherein two of said electrode pairs are comprised of three electrodes, one of said three electrodes being a member of two electrode pairs.

17. The apparatus of claim 14, wherein three of said electrode pairs are comprised of four electrodes, one of said four electrodes being a member of three electrode pairs.

18. The apparatus of claim 15, wherein said first members carry nine electrodes.

19. The apparatus of claim 15, wherein at least two of said electrode pairs stimulate the same nerves.

20. The apparatus of claim 15, wherein said electrode pairs of said first members comprise six electrode pairs for a first side of the face and said electrode pairs stimulate the Zygomatic, Temporal, Buccal, Mandibular, and Cervical nerve branches.

21. The apparatus of claim 20, wherein said electrode pairs of said first members stimulate the Zygomatic and Buccal nerve branches.

22. The apparatus of claim 21, wherein said electrode pairs of said first members stimulate the Mandibular and Cervical nerve branches.

23. The apparatus of claim 14, wherein each of the electrodes is a snap electrode having a post and each of the members has a plurality of openings that accommodate the post.

24. The apparatus of claim 23, wherein each of the members includes a net of synthetic material threads that form the openings.

25. An apparatus for treating a face, comprising:
a structure having at least one member attachable to the face; and
a plurality of electrodes, pairs of said electrodes being arranged with respect to predetermined facial points for stimulating the nerves of the face;
said electrodes including a shared electrode, said shared electrode being a member of more than one of said pairs, said shared electrode stimulating more than one of said facial points.

26. The apparatus of claim 25, wherein the shared electrode is placed over an insertion of a facial nerve that innervates a plurality of facial nerve branches.

27. The apparatus of claim 26, wherein the facial nerve branches include the Zygomatic, Temporal, Buccal, Mandibular, and Cervical nerve branches.

28. A treatment method, comprising:
positioning electrode pairs so as to stimulate predetermined nerves on a first body surface and a second body surface; and
alternately stimulating a first pair and a second pair of said electrode pairs, said first pair being located on the first surface and said second pair comprising a reciprocal electrode pair of said first pair and being located on the second surface, said first pair and said second pair being stimulated alternately for a first predetermined period of time.

29. The method of claim 28, wherein said first pair is stimulated a second predetermined period of time and, after said first pair is stimulated, said second pair is stimulated for a third predetermined period of time.

30. The method of claim 28, wherein after said first predetermined period of time has elapsed, a third pair and a fourth pair are alternately stimulated for a fourth predetermined period of time, said third pair comprising an electrode pair on the first surface and said fourth pair comprising a reciprocal electrode pair of said third pair and being located on the second surface.

31. The method of claim 30, wherein after said fourth period of time has elapsed, a fifth pair and a sixth pair are alternately stimulated for a fifth predetermined period of time, said fifth pair comprising an electrode pair on the first surface and said sixth pair comprising a reciprocal electrode pair of said fifth pair and being located on the second surface.

32. The method of claim 28, wherein said first predetermined period of time and said second predetermined period of time each comprise one second.

33. The method of claim 32, wherein said third predetermined period of time comprises three minutes.

34. The method of claim 28, wherein said electrode pairs of at least two of said electrode pairs share a common electrode.

35. The method of claim 28, wherein at least two pairs of said electrode pairs stimulate the same nerves.

36. The method of claim 35, wherein at least one pair of said electrode pairs stimulates more than one nerve.

37. An apparatus for treating a body, comprising:
a plurality of members shaped generally to the contour of a body surface; and
a plurality of electrode pairs, at least one of said electrode pairs being mounted on each of said members;
said members being connected to one another and adjustable with respect to one another so as to be conformable to the contours of the body surfaces of different users, at least one of said electrodes being mountable at each of a plurality of positions on said members.

38. The apparatus of claim 37, wherein said members comprise first members for a first body surface and second members for a second body surface.

39. The apparatus of claim 37, wherein two of said electrode pairs are comprised of three electrodes, one of said three electrodes being a member of two electrode pairs.

40. The apparatus of claim 37, wherein three of said electrode pairs are comprised of four electrodes, one of said four electrodes being a member of three electrode pairs.

41. The apparatus of claim 38, wherein said first members carry nine electrodes.

42. The apparatus of claim 38, wherein at least two of said electrode pairs stimulate the same nerves.

43. The apparatus of claim 38, wherein each of the electrodes is a snap electrode having a post and each of the members has a plurality of openings that accommodate the post.

44. The apparatus of claim 43, wherein each of the members includes a net of synthetic material threads that form the openings.

45. An apparatus for treating a body, comprising:
a structure having at least one member attachable to a body surface; and
a plurality of electrodes, pairs of said electrodes being arranged with respect to predetermined body surface points for stimulating the nerves of the body;
said electrodes including a shared electrode, said shared electrode being a member of more than one of said pairs, said shared electrode stimulating more than one of said body surface points.

46. The apparatus of claim 45, wherein the shared electrode is placed over an insertion of a nerve that innervates a plurality of nerve branches.

47. A treatment method, comprising:
positioning at least one set of electrode pairs so as to stimulate reciprocal nerves of the body, each electrode pair set having a first electrode pair and a second electrode pair, each electrode pair set being associated with a respective set of reciprocal nerves in that the first and second electrode pairs of the set are positioned to stimulate the respective set of reciprocal nerves, each electrode pair set being associated with a respective predetermined period of time; and
alternately stimulating the first pair and the second pair for the respective predetermined period of time.

48. The method of claim 47, comprising sequentially stimulating each electrode pair set, wherein stimulating each electrode pair set comprises alternately stimulating the first pair of the electrode pair set and the second pair of the electrode pair set for the predetermined period of time respective to the electrode pair set.

49. The method of claim 47, wherein each set of reciprocal nerves comprises facial nerves.

50. The method of claim 47, wherein at least one of the first electrode pair and the second electrode pair of at least one electrode pair set, and at least one of the first electrode pair and the second electrode pair of at least one other electrode pair set, share a common electrode.

51. The method of claim 47, wherein at least one of the first electrode pair and the second electrode pair of at least one electrode pair set, and at least one of the first electrode pair and the second electrode pair of at least one other electrode pair set, stimulate the same set of reciprocal nerves.
